(19) United States
(12) Patent Application Publication
(10) Pub. No.: US 2003/0119892 A1
(43) Pub. Date: Jun. 26, 2003

(54) Methods and compositions for treating headache pain with topical NSAID compositions

(76) Inventors: Larry Caldwell, San Jose, CA (US); Bradley S. Galer, West Chester, PA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/029,407

(22) Filed: Dec. 26, 2001

(51) Int. Cl. A61K 31/415; A61K 31/192

(52) U.S. Cl. 514/406; 514/570; 424/449

(57) ABSTRACT

Methods and compositions are provided for treating a host suffering from headache pain. In the subject methods, a topical NSAID formulation is applied to a keratinized skin site proximal to the pain associated with the headache pain, e.g., a keratinized skin surface of the head, such as the forehead, temple, etc. Practice of the subject methods results in at least a reduction in the intensity of the pain associated with the headache. The subject methods and compositions find use in the treatment of a variety of headache conditions.

### INTRODUCTION

1. Technical Field

The field of this invention is the treatment of headache pain.

2. Background of the Invention

Headaches are a common problem affecting a large segment of the population. Headaches, such as tension type and migraine headaches, occur both intermittently and chronically, and can arise in response to variety of stimulants, including stress, injury, toxins in the environment and the like.

A variety of therapeutic agents have been developed for use in the treatment of patients suffering from headache pain. Some agents, such as aspirin, acetaminophen, vasoconstrictors and NSAIDs, e.g. ibuprofen and naprosyn, are administered systemically. Despite the prevalence of this form of treatment for headache pain, in some cases, systemic administration is not recommended. For example, oral administration of aspirin can result in stomach upset and patient discomfort. Furthermore, the agent can exert host systemic toxicity which may outweigh any therapeutic benefits provided by the agent. Finally, since the agent is administered systemically, its effects are also systemic, which may not be desired.

Thus, there is continued interest in the identification of topical formulations that are suitable for use in the treatment of headache pain. Such topical formulations should provide for rapid penetration of an effective amount of the active agent through the skin surface, and thereby provide for rapid pain relief.

3. Relevant Literature

United States patents of interest include: U.S. Pat. Nos. 5,840,755; 5,776,952 and 5,667,799. Topical formulations of NSAIDS include those described in U.S. Pat. Nos. 4,670,254; 4,710,497; 4,740,374; 4,777,046; 4,956,171; 4,999,379; 5,204,119; 5,373,022; 5,374,661; 5,429,590; 5,695,779; 5,814,599; and EPB 0574255, the disclosures of which are herein incorporated by reference.

### SUMMARY OF THE INVENTION

Methods and compositions are provided for treating a host suffering from headache pain. In the subject methods, a topical NSAID formulation is applied to a keratinized skin site proximal to the pain associated with the headache pain, e.g., a keratinized skin surface of the head, such as the forehead, temple, occipital region, etc. Practice of the subject methods results in at least a reduction in the intensity of the pain associated with the headache. The subject methods and compositions find use in the treatment of a variety of headache conditions.

### DESCRIPTION OF THE SPECIFIC EMBODIMENTS

Methods and compositions are provided for treating a host suffering from headache pain. In the subject methods, a topical NSAID formulation is applied to a keratinized skin site proximal to the pain associated with the headache pain, e.g. a keratinized skin surface of the head, such as the forehead, temple, occipital region, etc. Practice of the subject methods results in at least a reduction in the intensity of the pain associated with the headache. The subject methods and compositions find use in the treatment of a variety of headache conditions. Also provided are kits for use in practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the components which are described in the publications which might be used in connection with the presently described invention.

Methods

As summarized above, the subject invention provides a method for treating a host suffering from a headache, i.e., from a pain in the head, cephalalgia, headache pain. The headache may be any of a variety of different types of headaches, including but not limited to: tension headache, migraine headache, cluster headache, headache associated with inflamed sinuses, temporal arteritis, cervicogenic, or other causes.

As indicated above, topical NSAID formulations are employed in the subject methods. In the broadest sense, any convenient topical formulation that provides for the requisite penetration of the NSAID in the formulation through the keratinized skin surface and to the target area of the host may be employed. The topical formulation may be a gel, lotion, cream, patch, hydrogel patch, aerosol or some other topical formulation. The topical formulation is one that does not include opioids. In many embodiments, the topical formulation is one in which the sole active agent is an NSAID.

Topical formulations of NSAIDS are known in the art, where suitable formulations include those described in: U.S. Pat. Nos. 4,670,254; 4,710,497; 4,740,374; 4,777,046; 4,956,171; 4,999,379; 5,204,119; 5,373,022; 5,374,661; 5,429,590; 5,695,779; 5,814,599; and EPB 0574255, the disclosures of which are herein incorporated by reference. Of particular interest in many embodiments are cream or patch formulations.

The NSAIDs used in the present compositions are well known in the pharmaceutical art, are prepared via methods well known in the chemical and pharmaceutical arts, and include, for example, pharmaceutically active compounds having at least one acid moiety wherein such acid moiety is, most preferably, a carboxylic acid. Other acid moieties are well known to one of ordinary skill in the art. Representative non-steroidal, anti-inflammatory drugs (NSAIDs) include, but are not limited to: diclofenac, fenoprofen, flurbiprofen, ibuprofen, indomethacin, ketoprofen, ketorolac, naproxen, sulindac, etodolac, tometin, diflunisal, mefenamic acid, meclofenamic acid, and flufenamic acid. Also of interest in certain embodiments are COX2 inhibitors, e.g., Celecoxib, Rofecoxib, etc. This list of NSAIDs is presented solely for the purpose of exemplification and is not intended to be limiting. Other NSAIDs which are used in the compositions of the present invention, are described, in addition to their dose regimens, in well known references such as, for example, the Physician's Desk Reference and the Merck Index. In many embodiments, the NSAID active agent present in the topical NSAID formulation is a nonsalycilate NSAID, where representative nonsalicylate NSAIDS include: propionic acids, e.g. fenoprofen, flurbiprofen, ibuprofen, ketoprofen, naproxen, etc.; acetic acids, e.g. diclofenac, etodolac, indomethacin, ketorolac, sulindac, tolmetin, etc.; fenamates, e.g. meclofenamate, mefenamic acid, etc.; oxicams, e.g. piroxicam, etc.; nabumetone; indomethacin; and the like.

The topical NSAID formulation employed in the subject methods is a formulation that includes an effective amount of an NSAID active agent, as described above. While the amount of any particular NSAID active agent may vary, in many embodiments, the amount of NSAID present in the topical formulation is one that is effective for at least reducing the pain associated with the syndrome being treated, and generally ranges from about 0.1 to about 5.0% w/w, usually from about 0.5 to about 3.0% w/w and more usually from about 0.5 to about 2.0% w/w.

In practicing the subject methods, the topical NSAID formulation is applied to a keratinized skin surface of the host in a manner sufficient to provide for penetration of an affective amount of the NSAID to the nerves and soft tissues, muscles, tendons, ligaments, etc., causing the pain associated with the syndrome being treated. In other words, the topical NSAID formulation is applied to the keratinized skin surface of the host at a region sufficiently proximal to the pain origin to provide for sufficient penetration of the NSAID through the keratinized skin surface and to pain origin. Typically, the topical formulation is applied to a keratinized skin surface of the head, where head keratinized skin surfaces of interest include the forehead and regions thereof, the temples, occipital region, neck, and the like. The topical formulation can be applied to one or more distinct regions, depending on the pain origin.

In certain embodiments of the subject methods, the topical composition comprising the NSAID is applied to a keratinized skin site of the host proximal to target nerves associated with the headache pain. Nerves which are commonly associated with headache pain are the occipital and supraorbital nerves. The skin site at which the composition is applied will be sufficiently proximal to the target nerves, e.g., the skin site overlies the region innervated by the target nerves, so that upon contact of the composition with the skin surface, the NSAID active agent can readily reach the target nerves (and/or muscles of the forehead, temples and occipital regions) and exert its anti-inflammatory activity. Of particular interest as skin sites of topical application are the supraorbital and occipital regions.

The amount of composition applied is one that is sufficient to provide for the desired reduction in pain intensity. The exact amount of topical composition that is applied may be determined empirically. For solutions, dispersions, gels, lotions, creams and the like, the composition will be spread over the region and a covering optionally applied thereto. For patches, an appropriate sized patch will be placed over the region comprising the skin site. Generally, the area of skin that is covered with topical formulations is at least about 1 cm², usually at least about 3 cm² and more usually at least about 6 cm² and may be as great as 10 cm² or greater, but typically does not exceed about 25 cm² and usually does not exceed about 15 cm².

The formulation is maintained in place for a period of time sufficient for the desired amelioration in symptoms, e.g., reduction in pain, to occur. Generally, the formulation is maintained in place for at least about 30 min, usually at least about 1 hr and more usually for at least about 4 hr, where the formulation may be maintained in place for as long as 8 hr, 12 hr or longer.

Upon application of the topical composition, the NSAID present therein penetrates the surface of the skin to reach the pain origin and reduce at least one symptom associated with the syndrome being treated. As such, application and maintenance of the topical NSAID formulation as described above results in at least an amelioration of at least one symptom associated with the syndrome being treated, e.g., a reduction in pain. By “at least an amelioration” is meant at least a reduction in the magnitude of the symptom, including a complete cessation or removal of the symptom. Typically, the symptom ameliorated by the subject methods is pain associated with the syndrome being treated. In many embodiments, the intensity of pain associated with the syndrome is at least reduced, if not eliminated. Reduction in pain intensity is evaluated according to the well known Pain Relief Score protocol (where 0-worse pain; 1-no change; 2-slight improvement; 3-moderate improvement; 4-alot of improvement; 5-complete relief). The magnitude of reduction in pain intensity is generally at least about 10% (slight relief), usually at least about 25% (slight-moderate relief) and more usually at least about 50% (moderate relief), where magnitude of reduction may be as high as 75%, 80%, 95% or higher (alot of relief), including a complete cessation of pain (complete relief).

A feature of the subject methods is that they result in no or substantially no side toxic side effects which are observed in systemic, e.g., oral, NSAID delivery mechanisms. As such, the subject methods results in no or substantially no nausea, vomiting, etc., as observed with other systemically administered NSAIDs.

Treatment according to the subject methods results in at least amelioration from one or more symptoms of the underlying condition, as described above, for a period of time of at least about 3 hrs, usually at least about 6 hrs and more usually at least about 12 hrs or longer, e.g., 16 hrs, 24 hrs, or longer.

Utility

The above described invention finds use in treating a host suffering from a headache, i.e., from a pain in the head, cephalalgia, headache pain. The headache may be any of a variety of different types of headaches, including but not limited to: tension headache, migraine headache, cluster headache, headache associated with inflamed sinuses, temporal arteritis, or other causes.

A variety of hosts are treatable according to the subject methods. Generally such hosts are “mammals” or “mammalian,” where these terms are used broadly to describe organisms which are within the class mammalia. Of particular interest is the treatment of primates with the subject methods, (e.g., humans, chimpanzees, and monkeys), where the subject methods are particularly suited for use in the treatment of humans suffering from indomethacin responsive headache syndromes, as described above.

Topical Pharmaceutical Compositions

Also provided are topical pharmaceutical compositions comprising an effective amount of an NSAID active agent as described above, where the topical composition is present in a configuration that is tailored for its use in the treatment of headache pain according to the subject methods. For example, topical patch formulations and analogous structures are provided that are shaped specifically with respect to the target epidermal location of their intended application, e.g., to cover the requisite surface area of the target location as described above, e.g., as rectangular, square, round, oval or other shapes configured to cover a forehead or temple target skin surface in a manner described above. The amount of active NSAID agent present in the formulation may vary depending on the nature of the formulation and the specific NSAID active agent, but in many embodiments ranges from about 0.1 to about 5.0%, usually from about 0.5 to about 3.0%, and more usually from about 0.5 to about 2.0% w/w.

Kits

Kits with NSAID topical formulations used in the subject methods, are provided.

Conveniently, the topical formulations may be provided in a unit dosage format, which formats are known in the art. In such kits, in addition to the containers containing the formulation, e.g. unit doses, is an informational package insert describing the use of the subject formulations in the methods of the subject invention, i.e. instructions for using the subject unit doses to treat headache pain. Theses instructions may be present on one or more of the packaging, a package insert, and the like.

The following examples are offered by way of illustration and not by way of limitation.

### Experimental

A 38 year old woman with a history of migraine headaches suddenly developed a severe frontal throbbing headache associated with severe light sensitivity and nausea. Past attacks have lasted up to several hours and were characterized by severe pain, light sensitivity and nausea. Because her nausea was so severe, she was not able to take any medication by mouth (orally). Approximately 10 minutes into her migraine attack, she applied a topical patch containing diclofenac as the active agent (tape type) containing the active ingredient directly to her forehead and temples. Within 15 minutes, she began to report a mild to moderate degree of relief from her headache pain, light sensitivity and nausea. Within 30 minutes, she described a moderate to fair amount of relief from her headache pain, light sensitivity and nausea. Within 45 minutes, she described complete relief from her headache pain, light sensitivity and nausea. She stated the relief was faster and more complete than she has experienced from oral NSAIDs and oral acetominophen.

No side effects were reported from treatment with the NSAID patch.

It is evident from the above results and discussion that improved methods of treating disease conditions associated with headache pain are provided. The subject methods offer a convenient, non-surgical form of treatment that nonetheless provides for rapid amelioration of at least one symptom associated with the disease condition being treated. Furthermore, the subject methods are amenable to self-administration and do not give rise to systemic side effects, since the active agent only acts locally. As such, the subject invention provides for a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for at least ameliorating headache pain of a host, said method comprising:
topically applying an affective amount of a topical NSAID formulation to a keratinized skin surface of said host to at least ameliorate said headache pain.

2. The method according to claim 1, wherein said topical NSAID formulation comprises a nonsalicylate NSAID.

3. The method according to claim 1, wherein said topical formulation is a cream.

4. The method according to claim 1, wherein said topical formulation is a patch.

5. The method according to claim 1, wherein said host is a mammal.

6. A method of treating a mammal suffering from headache pain, said method comprising:
topically applying a nonsalicylate NSAID formulation to a keratinized skin surface of said mammal for a period of time sufficient for amelioration of said headache pain to occur.

7. The method according to claim 6, wherein said mammal is a human.

8. The method according to claim 6, wherein said formulation is a cream.

9. The method according to claim 6, wherein said formulation is a patch.

10. The method according to claim 6, wherein said keratinized skin surface is selected from the group consisting of the forehead, temple and/or occipital region.

11. A method for treating a human suffering from headache pain, said method comprising:
topically applying a nonsalicylate NSAID formulation to at least one of the forehead, temples and/or occipital region of said human to at least reduce said pain.

12. The method according to claim 11, wherein said NSAID formulation is a cream.

13. The method according to claim 11, wherein said NSAID formulation is a patch.

14. The method according to claim 11, wherein said NSAID is an acetic acid.

15. The method according to claim 14, wherein said NSAID is diclofenac.

16. The method according to claim 11, wherein said NSAID is indomethacin

17. The method according to claim 11, wherein said NSAID is ibuprofen.

18. The method according to claim 14, wherein said NSAID is ketoprofen.

19. A kit for use in treating a subject suffering from headache pain, said kit comprising:
a topical NSAID formulation; and
instructions for practicing the method according to claim 1.

20. The kit according to claim 19, wherein said NSAID formulation comprises a nonsalicylate NSAID.

21. The kit according to claim 19, wherein said formulation is a cream.

22. The kit according to claim 19, wherein said formulation is a patch.

23. The kit according to claim 19, wherein said NSAID is an acetic acid.
